(19) United States
(12) Patent Application Publication
(10) Pub. No.: US 2013/0045280 A1
(43) Pub. Date: Feb. 21, 2013

(54) PRODUCT AND TREATMENT FOR HEPATITIS C AND SCLEROSIS OF THE LIVER AND METHODS OF FORMING THE PRODUCT

(71) Applicant: Robert Radin, Las Vegas, NV (US)

(72) Inventor: Robert Radin, Las Vegas, NV (US)

(21) Appl. No.: 13/210,058

(22) Filed: Aug. 15, 2011

(51) Int. Cl. A61K 35/12; A61P 1/16; A61P 31/14

(52) U.S. Cl. 424/543

(57) ABSTRACT

A method for formulating a treatment for hepatitis C and sclerosis of the liver is disclosed. The method may comprise steps of processing bone marrow of a bone of a bird. To prepare the composition for feeding to a mammal, the mammal is prepared by purging the mammal's intestines of fecal matter. After purging the mammal's intestines of fecal matter, the processed bird bone is fed to the mammal. The mammal's feces is then collected and mixed with crushed pumpkin seed.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates to a product and treatment for hepatitis C and/or sclerosis of the liver and a method for formulating the product.

Approximately 180 million people worldwide are infected with Hepatitis C. Hepatitis C is an infectious disease affecting the liver. Hepatitis C is asymptomatic but chronic infection can cause scarring of the liver or even liver failure. Hepatitis C can be treated with a cure rate of about 51%. However, there is no currently available vaccine for hepatitis C.

Hepatitis C also causes other health conditions such as sclerosis of the liver. The healthy tissues of the liver is slowly replaced with scarring tissue through chronic hepatitis C infections. Also, according to the National Institute of Health, sclerosis of the liver is the 12^{th} leading cause of death by disease.

For the foregoing reasons, there is a need for additional treatments for hepatitis C and/or sclerosis of the liver.

### BRIEF SUMMARY

The product, the method for forming the product and the treatment disclosed herein addresses the needs discussed above, discussed below and those known in the art.

The method for formulating the product to treat Hepatitis C and/or sclerosis of the liver includes the steps of preparing a food product, purging intestines of a mammal, feeding the prepared food product to the mammal, collecting feces of the mammal and mixing pumpkin seeds with the feces.

More particularly, the method of formulating the product for the treatment of hepatitis C or sclerosis of the liver may comprise the steps of cooking bones of a bird; grinding the cooked bird bones into a powder; adding water to the ground and cooked bird bones to form a paste; purging a mammal's intestine of fecal matter; after the purging step, feeding the paste to the mammal; collecting white speckled feces of the mammal; drying the white speckled feces; crushing the dried white speckled feces into a powder; and adding crushed pumpkin seeds to the crushed and dried white speckled feces.

The cooking step may include the step of cooking bones of a chicken. Also, the cooking step may include the step of extracting marrow of a chicken and cooking the bone marrow of the chicken.

The purging step may include the step of inducing a fast in the mammal. The fast may last for one or more days.

The drying step may include the step of exposing the white speckled feces to sun light.

The adding step may include the step of adding about 6 to about 10 wt. % crushed pumpkin seed to the crushed and dried white speckled feces.

Moreover, a product is disclosed which is formulated by the method discussed herein.

Additionally, a treatment for the treatment of hepatitis C or sclerosis of the liver is disclosed. The treatment may comprise the steps of providing a product produced by the process of cooking bones of a bird; grinding the cooked bird bones into a powder; adding water to the ground and cooked bird bones to form a paste; purging a mammal's intestine of feces; after the purging step, feeding, the paste to the mammal; collecting white speckled feces of the mammal; drying the white speckled feces; crushing the dried white speckled feces into a powder; and adding crushed pumpkin seeds to the crushed and dried white speckled feces.

The treatment method may also include the step of administering the product to a person having hepatitis C or sclerosis of the liver.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a flowchart of the method of formulating the treatment.

### DETAILED DESCRIPTION

The product, method for formulating the product and treatment discussed herein relate to a treatment for hepatitis C and/or sclerosis of the liver. In formulating the product, the bone of a bird (e.g., chicken) is processed for feeding to a mammal (e.g., dog). Before feeding the processed bird bone, the mammal is placed on a fast to purge the mammal of any fecal matter. After the mammal's intestines are purged, the mammal is fed the processed bird bone. The mammal's feces will be collected which will have a white speckled consistency. Any feces having a consistency other than a white speckled consistency is separated and discarded. The mammal's feces having the white speckled consistency is processed and administered to a person having hepatitis C and/or sclerosis of the liver.

The treatment disclosed herein starts with the bone of a bird. The bone may be that of any type of bird including but not limited to poultry, game fowl, and water fowl. Preferably, the bird is a chicken but can include other birds or bird combinations. The bones and preferably the bone marrow of the chicken are cooked 100. By way of example and not limitation, the chicken bone may be cooked 100 by steaming, roasting, microwaving, baking, etc. In all instances, the chicken bone is raised to a predetermined temperature and maintained at such temperature for a predetermined period of time to cook the chicken bone. Preferably, the marrow of the chicken bone is extracted and cooked through conventional means discussed herein, known in the art or developed in the future.

After cooking the chicken bone, the chicken bone may be ground down 102 into a powder form. The grinding 102 may be conducted with an industrial grade food grinding machine or by hand as is known in the art or developed in the future. The chicken bone may be ground 102 until it generally has a powdery consistency.

After grinding the chicken bone, the ground chicken bone is now mixed with water 104 to form a pasty consistency.

The chicken bone paste is then fed 106 to a mammal. Prior to feeding the mammal, the intestines of the mammal must be purged 108 of any feces. By way of example and not limitation, the mammal may be placed on a fast and allowed to excrete any fecal matter. When the mammal stops excreting fecal matter for a predetermined period of time depending on the mammal, one can be reasonably sure that most if not all of the mammal's fecal matter has been purged from the mammal. By way of example and not limitation, the mammal may be a dog. However, other mammals are also contemplated such as cat, sheep, etc. The dog may be placed on a fast for one or more days until the dog's intestines are clear of any fecal matter. After purging 108 the dog's intestines, the dog is fed 106 the chicken bone paste. When the dog excretes fecal matter, the dog's feces should have a white speckled consistency or transition to a white speckled consistency shortly thereafter. If not, then the dog must be placed on the fast for a longer period of time so that the chicken bone paste can show up in the dog's feces.

The dog's feces is collected 109 and processed. In particular, the dog's feces may have various consistencies especially if all of the dog's feces was not fully purged through the fast. Eventually, the dog's fecal waste product will have a white speckled consistency. Any feces having a consistency other than a white speckled consistency is separated and discarded. Only the portion of the feces having the white speckled consistency is utilized. The portion of the feces having the white speckled consistency may be separated from other portions of the feces having a different consistency by hand or machine.

The white speckled feces may then be dried 110. In particular, the moisture content of the white speckled feces may be reduced to a predetermined level. By way of example and not limitation, the white speckled feces may be dried 110 in the sun (i.e., solar drying) if the air is sufficiently hot and dry or in an oven or dryer.

The dried white speckled feces is then crushed 112. Crushed pumpkin seed is mixed 114 with crushed white speckled feces. By way of example and not limitation, the crushed pumpkin seed may be about 3 to about 15 wt. percent of the dried and crushed white speckled feces. Preferably, about 6 to about 10 wt. % crushed pumpkin seed is mixed 114 with the dried and crushed white speckled feces to form the product.

The product may be administered to an adult or a child. For an adult, 1 tablespoon, twice a day for four to six months of the product may be ingested by the person. The dosage of a child is about ½ the dosage for the adult.

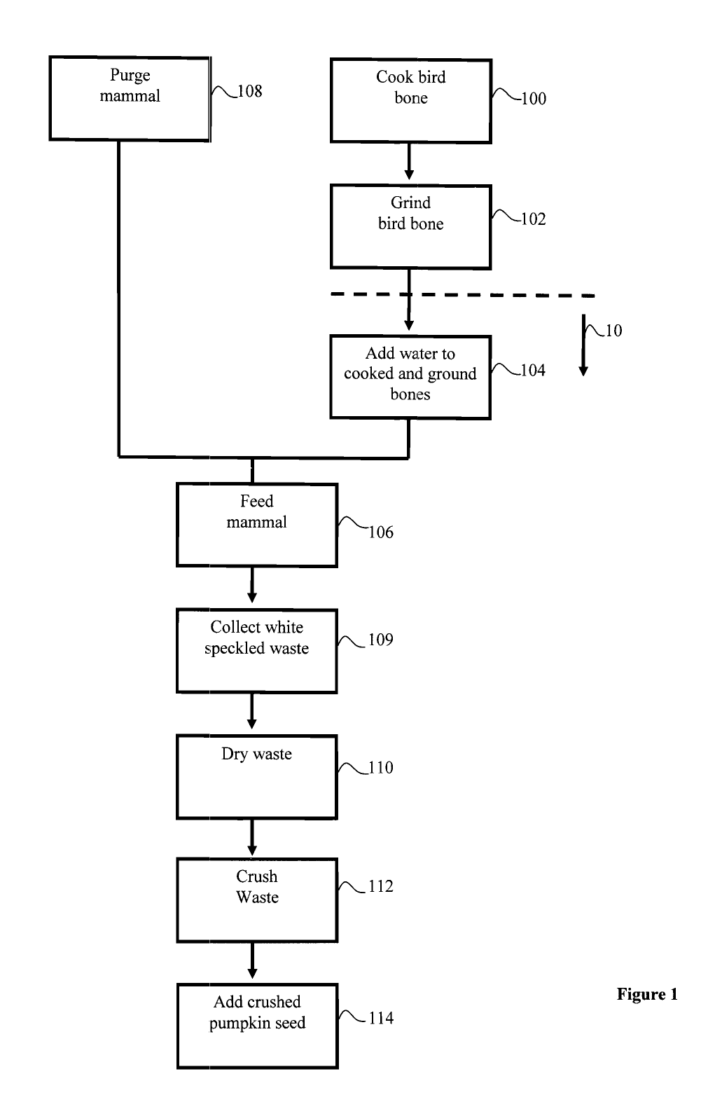

In an aspect of the treatment, it is also contemplated that the cooked and ground bird bone may be provided to a person that will complete the rest of the steps of formulating the product. In particular, the person will the perform the other steps of formulating the product (see arrow 10 shown in FIG. 1) including but not limited to adding 104 water to the cooked and ground bird bone, purging 108 the mammal's intestine, feeding 106 the pasty cooked and ground bird bone to the mammal, collecting 109 the white speckled feces, drying 110 and crushing 112 the white speckled feces and mixing 114 in crushed pumpkin seed with the dried and crushed white speckled feces.

The above description is given by way of example, and not limitation. Given the above disclosure, one skilled in the art could devise variations that are within the scope and spirit of the invention disclosed herein, including various dosage amounts. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of formulating a product for the treatment of hepatitis C or sclerosis of the liver, the method comprising the steps of:
cooking bones of a bird;
grinding the cooked bird bones into a powder;
adding water to the ground and cooked bird bones to form a paste;
purging a mammal's intestine of fecal matter;
after the purging step, feeding the paste to the mammal;
collecting white speckled feces of the mammal;
drying the white speckled feces;
crushing the dried white speckled feces into a powder;
adding crushed pumpkin seeds to the crushed and dried white speckled feces.

2. The method of claim 1 wherein the cooking step includes the step of cooking bones of a chicken.

3. The method of claim 2 wherein the cooking step includes the step of extracting marrow of a chicken and cooking the bone marrow of the chicken.

4. The method of claim 1 wherein the purging step includes the step of inducing a fast in the mammal.

5. The method of claim 4 wherein the fast is for one or more days.

6. The method of claim 1 wherein the drying step includes the step of exposing the white speckled feces to sun light.

7. The method of claim 1 wherein the adding step includes the step of adding about 6 to about 10 wt. % crushed pumpkin seed to the crushed and dried white speckled feces.

8. A product formulated by the method of claim 1.

9. A treatment for the treatment of hepatitis C or sclerosis of the liver, the treatment comprising the steps of:
providing a product produced by the process of:
cooking bones of a bird;
grinding the cooked bird bones into a powder;
adding water to the ground and cooked bird bones to form a paste;
purging a mammal's intestine of fecal matter;
after the purging step, feeding the paste to the mammal;
collecting white speckled feces of the mammal;
drying the white speckled feces;
crushing the dried white speckled feces into a powder;
adding crushed pumpkin seeds to the crushed and dried white speckled feces;
administering the product to a person having hepatitis C or sclerosis of the liver.
